(19) United States
(12) Patent Application Publication
(10) Pub. No.: US 2007/0116785 A1
(43) Pub. Date: May 24, 2007

(54) Nitric oxide as an anti-viral agent, vaccine and vaccine adjuvant

(76) Inventor: Christopher C. Miller, North Vancouver (CA)

Correspondence Address: SIDLEY AUSTIN BROWN & WOOD LLP (LAIP GROUP), 555 W. FIFTH ST., SUITE 4000, LOS ANGELES, CA, 90013, US

(21) Appl. No.: 11/601,908

(22) Filed: Nov. 20, 2006

Related U.S. Application Data
(60) Provisional application No. 60/737,997, filed on Nov. 18, 2005.

(51) Int. Cl. A61K 33/00

(52) U.S. Cl. 424/718

(57) ABSTRACT

The invention relates to uses of nitric oxide gas to inactivate any whole, part or subunit of a microbe, such as a virus. Such an inactivated or attenuated virus after treatment with NO gas may be used in formulations for a vaccine. Nitric oxide may be administered directly to create a vaccine through in vitro and/or in situ or by direct administration in vivo. Also provided are methods for treating patients with viral infections through the inhalation of nitric oxide gas.

### CLAIM OF PRIORITY

This application claims priority to U.S. Provisional Application Ser. No. 60/737,997 filed on Nov. 18, 2005, which is expressly incorporated herein by reference in its entirety.

### TECHNICAL FIELD

The present invention relates to the treatment of viruses, including the treatment of subjects having viral infections and the use and manufacture of viral vaccines.

### BACKGROUND ART

Viral vaccines are traditionally of two sorts. The first sort are “killed” or “inactivated” vaccines, which are virus preparations which have been killed by treatment with a suitable chemical such as beta-propriolactone. The second type are live “attenuated” or “weakened” vaccines, which are viruses which have been rendered less pathogenic to the host, either by specific genetic manipulation of the virus genome, or, more usually, by passage in some type of tissue culture system. These two types of vaccine each have their own advantages and disadvantages.

Production of viral vaccines has been described for both inactivated and attenuated viral vaccines. The methods generally involve growing a cell culture, treating the virus with the anti-viral agent to inactivate or attenuate it, and formulating into a vaccine with a typical pharmaceutical carrier. Several patents describe these methods, including U.S. Pat. Nos. 6,344,354; 6,306,637; 5,948,410; 5,837,261; 5,824,536; 5,665,362; 5,639,461; 4,692,412; 4,525,349; 4,318,903; 3,959,074; and 3,933,585, each herein incorporated by reference.

The flu virus has been an intensive topic of scientific research and in the popular media of recent years. The highly pathogenic avian influenza (HPAI) strain H5N1 has had scientists and government attention since it first appearance in 1997. Serious concerns regarding viral pathogens are easily justified as their ease of spread, coupled with their virulence, their ability to mutate and lack of effective therapeutic agents, make containment and treatment a worldwide health care challenge. HPAI H7N3 is also of serious concern to the scientific community and is similar to H7N1 as it is an influenza type A also.

Nitric oxide (NO) is an environmental pollutant produced as a byproduct of combustion. At extremely high concentrations (generally at or above 1000 ppm), NO is toxic. NO also is a naturally occurring gas that is produced by the endothelium tissue of the vascular system. In the 1980's, it was discovered by researchers that the endothelium tissue of the human body produced NO, and that NO is an endogenous vasodilator, namely, an agent that widens the internal diameter of blood vessels.

With this discovery, numerous researchers have investigated the use of low concentrations of exogenously inhaled NO to treat various pulmonary diseases in human patients. See e.g., Higenbottam et al., Am. Rev. Resp. Dis. Suppl. 137:107, 1988. It was determined, for example, that primary pulmonary hypertension (PPH) can be treated by inhalation of low concentrations of gaseous NO (gNO). With respect to pulmonary hypertension, inhaled NO has been found to decrease pulmonary artery pressure (PAP) as well as pulmonary vascular resistance (PVR). The use of inhaled NO for PPH patients was followed by the use of inhaled NO for other respiratory diseases. For example, NO has been investigated for the treatment of patients with increased airway resistance as a result of emphysema, chronic bronchitis, asthma, adult respiratory distress syndrome (ARDS), and chronic obstructive pulmonary disease, (COPD). In 1999, the FDA approved the marketing of nitric oxide gas for use with persistent pulmonary hypertension in term and near term newborns. Because the withdrawal of inhaled nitric oxide from the breathing gas of patients with pulmonary hypertension is known to cause a severe and dangerous increase in PVR, referred to as a “rebound effect”, nitric oxide must be delivered to these patients on a continuous basis.

In addition to its effects on pulmonary vasculature, NO may also be introduced as an anti-microbial agent against pathogens via inhalation or by topical application. See e.g., WO 00/30659, U.S. Pat. No. 6,432,077, which are hereby incorporate by reference in their entirety. The application of gaseous nitric oxide to inhibit or kill pathogens is thought to be beneficial given the rise of numerous antibiotic resistant bacteria. For example, patients with pneumonia or tuberculosis may not respond to antibiotics given the rise of antibiotic resistant strains associated with these conditions.

It has been found that the treatment of a virus with nitric oxide gas may inactive or attenuate the virus, and that such treated virus may be used in formulating a vaccine.

### SUMMARY OF THE INVENTION

A first embodiment of the invention is a method of inactivating or attenuating a virus comprising: (1) identifying a virus; and (2) exposing the virus to nitric oxide gas for a period of time and at a concentration sufficient to inactivate or attenuate the virus. This method produces a treated virus that may be used in formulating vaccines or vaccine adjuvants. This method also contemplates that the virus may be directly contacted within a patient.

In the various methods of the present invention, the exposure step may be accomplished in vivo or in vitro. The in vivo step may be accomplished through inhalation of the nitric oxide gas into the respiratory tract of a patient. The period of time may be as short as 5-10 minutes (approximately 100 breathes per minute) with a single dose or at least about 20 minutes or greater, such as from about 30 minutes every 4 hours or continuous to about 3 hours. The concentration may be about 120 ppm to about 400 ppm nitric oxide, preferably about 160 ppm. The viruses that may be inactivated or attenuated by the method include Influenza A, Influenza B, Avian Flu viruses, SARS (Corona) viruses, respiratory syncytial viruses, para-influenza viruses, Bovine Virus Diarrhea, HIV, and Rhinoviruses. Once inactivated, the treated virus may be formulated with one or more of a vaccine, an anti-viral agent, a vaccine adjuvant, an anti-viral adjuvant, nitric oxide, and a nitric oxide releasing compound.

Another embodiment of the present invention is a method of producing a treated virus for use as a vaccine or anti-viral agent comprising: (1) identifying a virus; and (2) exposing the virus to nitric oxide gas for a period of time and at a concentration sufficient to inactivate or attenuate the virus.

Another embodiment of the invention is a method of producing a vaccine comprising: (1) identifying a virus; (2) exposing the virus to nitric oxide gas for a period of time and at a concentration sufficient to inactivate or attenuate the virus; (3) adding the exposed virus to patient cells; (4) growing the infected and exposed patient cells; and (5) formulating the resulting grown cells into a vaccine. Alternatively, a method of producing a vaccine may include: (1) identifying a virus; (2) adding the virus to cultured patient cells; (3) exposing the infected cultured patient cells to nitric oxide gas for a period of time and at a concentration sufficient to inactivate or attenuate the virus; (4) growing the patient cells; and (5) formulating the resulting grown cells into a vaccine.

Another embodiment of the invention is a vaccine created by the above described methods.

Another embodiment of the invention is a vaccine comprising: (1) a pharmaceutically acceptable carrier or diluent; and (2) an exposed virus that is inactivated or attenuated, wherein the exposed virus is obtained by: (a) identifying a virus; and (b) exposing the virus to nitric oxide gas for a period of time and at a concentration sufficient to inactivate or attenuate the virus. The vaccine may also comprise one or more of another vaccine, an anti-viral agent, a vaccine adjuvant, an anti-viral adjuvant, nitric oxide, and a nitric oxide releasing compound.

Another embodiment of the invention is a method of treating a patient comprising: (1) providing a vaccine to a patient, wherein the vaccine comprises: (a) an exposed virus that is inactivated or attenuated, wherein the exposed virus is obtained by: (i) identifying a virus; and (ii) exposing the virus to nitric oxide gas for a period of time and at a concentration sufficient to inactivate or attenuate the virus. The patient may or may not have a viral infection or be exhibiting viral infection symptoms.

In all methods of treating patients described herein, the steps may include a step of administering a vaccine, an anti-viral agent, a vaccine adjuvant, an anti-viral adjuvant, nitric oxide, and a nitric oxide releasing compound to the patient.

Another embodiment of the invention is a method of treating a patient comprising: (1) providing a vaccine to a patient; and (2) administering nitric oxide gas through inhalation to the patient. The patient may or may not have a viral infection or be exhibiting viral infection symptoms.

Another embodiment of the invention is a method of treating a patient comprising: (1) providing an anti-viral agent to a patient; and (2) administering nitric oxide gas through inhalation to the patient. The patient may or may not have a viral infection or be exhibiting viral infection symptoms.

Another embodiment of the invention is a method of treating a patient with a viral infection comprising: (1) administering nitric oxide gas at a concentration of at least about 100 ppm to the patient. Another embodiment of the invention is a method of preventing viral infection in a patient comprising: (1) administering nitric oxide gas at a concentration of at least about 100 ppm to the patient. The administering step may be accomplished through inhalation of the nitric oxide gas into the respiratory tract of a patient. The administering time may be as short as 5-10 minutes (approximately 100 breathes per minute) with a single dose or at least about 20 minutes or greater, such as from about 30 minutes every 4 hours or continuous to about 3 hours. The concentration may be about 120 ppm to about 400 ppm nitric oxide, preferably about 160 ppm. The viruses that may be inactivated or attenuated by the method include Influenza A, Influenza B, Avian Flu viruses, SARS (Corona) viruses, respiratory syncytial viruses, para-influenza viruses, Bovine Virus Diarrhea, HIV, and Rhinoviruses.

Furthermore, the vaccines produced herein may be used to treat or protect a patient from viral infection. Additional methods of treatment include providing a vaccine to a patient in combination with inhaled nitric oxide gas. The vaccine may be formulated with one or more of nitric oxide gas, a NO releasing compound, a vaccine adjuvant, and an anti-viral adjuvant.

### BRIEF DESCRIPTION OF DRAWINGS

FIG. 1, corresponding to Example 1, illustrates 12 wells of MDCK cells, 6 wells on the left being the treated wells and the 6 wells on the right being the air control wells, wherein exposure of influenza A virus to 160 ppm nitric oxide gas for 1-3 hours reduces their virulence within host cells.

FIG. 2, corresponding to Example 2, illustrates 12 wells of MDCK cells, 6 wells on the left being the air control wells and the 6 wells on the right being the treated wells, wherein exposure of influenza A/H3N2 virus to 160 ppm nitric oxide gas for 1-3 hours reduces their virulence within host cells.

FIG. 3, corresponding to Example 2, illustrates the infectivity of influenza A/H3N2 virus over 6 continuous hours of exposure to 160 ppm nitric oxide gas.

FIG. 4, corresponding to Example 2, illustrates the infectivity of influenza A/H3N2 virus over 4 continuous hours of exposure to 80 ppm nitric oxide gas.

FIG. 5, corresponding to Example 2, illustrates the infectivity of influenza A/H3N2 virus over 20 hours after a single 30 minute exposure to 160 ppm nitric oxide gas.

FIG. 6, corresponding to Example 2, illustrates the infectivity of influenza A/H3N2 virus (MDCK cells inoculated with virus prior to gNO exposure) over 6 continuous hours of exposure to 160 ppm nitric oxide gas.

FIG. 7, corresponding to Example 2, illustrates the infectivity of influenza A/Victoria/H3N2 virus over 4 continuous hours of exposure to 80 ppm nitric oxide gas.

FIG. 8, corresponding to Example 2, illustrates the infectivity of influenza A/Victoria/H3N2 virus over 4 continuous hours of exposure to 160 ppm nitric oxide gas.

FIG. 9, corresponding to Example 2, illustrates the infectivity of influenza A/Victoria/H3N2 virus over 4 hours after a single 30 minute exposure to 160 ppm nitric oxide gas.

FIG. 10, corresponding to Example 2, illustrates the infectivity of influenza A/Victoria/H3N2 virus over 4 continuous hours of exposure to 40 ppm and 80 ppm nitric oxide gas.

FIG. 11, corresponding to Example 2, illustrates the infectivity of influenza A/Victoria/H3N2 virus over 4 continuous hours of exposure to 160 ppm and 800 ppm nitric oxide gas.

FIG. 12, corresponding to Example 2, compares several dosing techniques of 160 ppm gNO to determine the infectivity of influenza A/Victoria/H3N2 virus after exposure.

FIG. 13, corresponding to Example 3, illustrates the infectivity of Highly Pathogenic Avian Influenza (HPAI) H7N3 over 3 continuous hours of exposure to 160 ppm nitric oxide gas.

FIG. 14, corresponding to Example 4, illustrates the infectivity of Highly Pathogenic Avian Influenza (HPAI) H7N3 over 3 hours after a single 30 minute exposure to 160 ppm nitric oxide gas.

FIG. 15, corresponding to Example 5, compares the mean clinical scores of several groups of bovine subjects with Bovine Respiratory Disease after exposure to 160 ppm gNO.

FIG. 16, corresponding to Example 5, compares the IRT Temperatures of several groups of bovine subjects with Bovine Respiratory Disease after exposure to 160 ppm gNO.

FIG. 17, corresponding to Example 5, compares the clinical scores of two groups (Prophylactic and IRT Early Detection) of bovine subjects with Bovine Respiratory Disease after exposure to 160 ppm gNO.

FIG. 18, corresponding to Example 5, compares the IRT Temperature of two groups (Prophylactic and IRT Early Detection) of bovine subjects with Bovine Respiratory Disease after exposure to 160 ppm gNO.

### BEST MODE FOR CARRYING OUT THE INVENTION

Before the present compositions and methods are described, it is to be understood that this invention is not limited to the particular devices, compositions, methodologies or protocols described, as these may vary. It is also to be understood that the terminology used in the description is for the purpose of describing the particular versions or embodiments only, and is not intended to limit the scope of the present invention which will be limited only by the appended claims. For simplicity, each reference referred to herein shall be deemed expressly incorporated by reference in its entirety as if fully set forth herein.

Although any methods, devices, and materials similar or equivalent to those described herein can be used in the practice or testing of embodiments of the present invention, the preferred methods, devices, and materials are now described. All publications mentioned herein are incorporated by reference. Nothing herein is to be construed as an admission that the invention is not entitled to antedate such disclosure by virtue of prior invention. As used herein, terms such as “subject” and “patient” may be used interchangeable and is not limited to human, bovine, equine, and other animal subjects.

The invention relates to uses of nitric oxide gas to inactivate any whole, part or subunit of a microbe, such as a virus. Such an inactivated or attenuated virus after treatment with NO gas may be used in formulations for a vaccine. Nitric oxide may be administered directly to create a vaccine through in vitro and/or in situ or by direct administration in vivo. Also provided are methods for treating patients with viral infections through the inhalation of nitric oxide gas.

### EXAMPLE 1

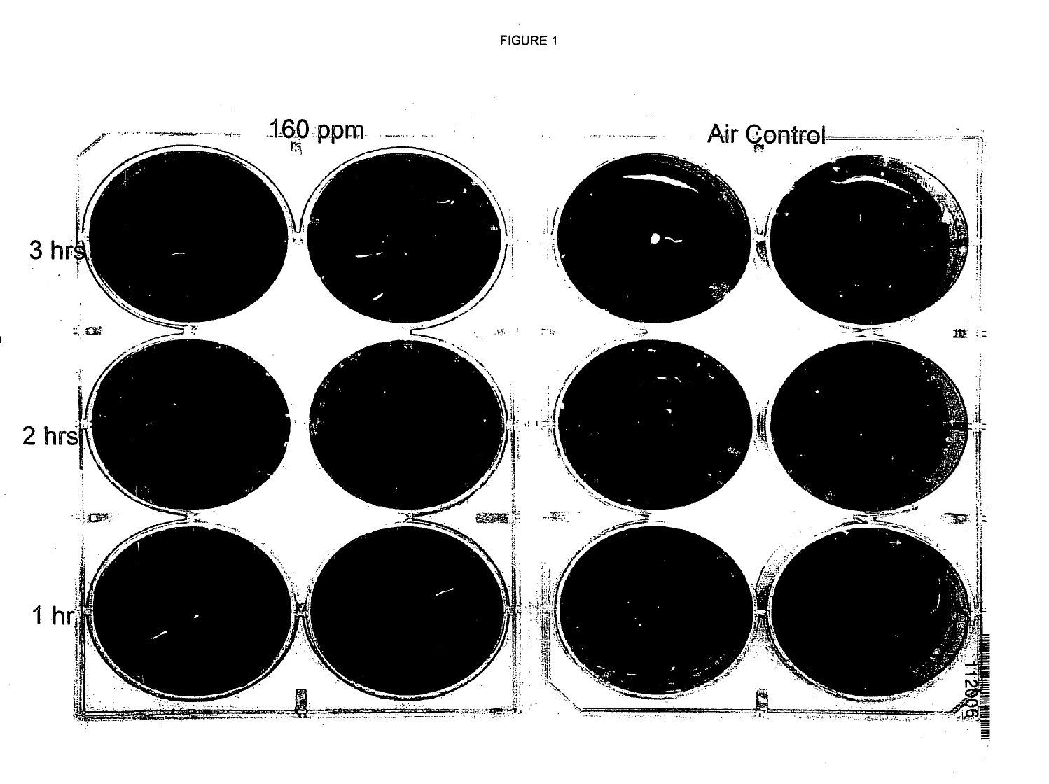

FIG. 1 illustrates that exposure of influenza A virus to 160 ppm nitric oxide gas for 1-3 hours reduces their virulence within host epithelial cells. First, 3 wells of influenza A virus (3 wells of 6×10⁵ pfu/mL) were exposed to 160 ppm nitric oxide gas for 1, 2, and 3 hours, respectively. FIG. 1 illustrates 12 wells of MDCK cells, 6 wells on the left being the treated wells and the 6 wells on the right being the air control wells, wherein exposure of influenza A virus to 160 ppm nitric oxide gas for 1-3 hours reduces their virulence within host cells. From the top to the bottom, the first row is exposure at 3 hours, while the bottom row is exposure at 1 hour.

Briefly, the experimental methods were as follows. Inoculums of influenza A virus were prepared to a suspension of 6×10⁵ pfu/mL, and diluted 1:1000 in sterile normal saline. Three milliliters of the inoculums were used per well in a sterile culture place. Exposure of the inoculums were performed in an exposure chamber, which has been described for example, in A. Ghaffari, D. H. Neil, A. Ardakani, J. Road, A. Ghahary, C. C. Miller, “A direct nitric oxide gas delivery system for the bacterial and mammalian cell cultures,” Nitric Oxide 12(3): 129-140 (May 2005), which is hereby incorporated by reference as if fully set forth herein. The inoculums were exposed to 160 ppm of NO gas at a flow rate of 2.5 liters per minute for each of 1, 2, and 3 hours.

From each exposed well, 1 mL of the exposed virus was extracted and frozen at −70° C. Additionally, influenza A virus was exposed for 1, 2, and 3 hours with air for control. Next, 6 wells of MDCK (canine kidney) cells were grown to a confluent monolayer. Once grown, 0.5 mL of 1 hour exposed virus was added to two wells of cultured cells, 0.5 mL of 2 hour exposed virus was added to two wells, and 0.5 mL of 3 hour exposed virus was added to two wells. Once combined, each cultured cell well incubated on a shaker well for 1 hour at 37° C. The 6 wells were then fixed with agar/media/trypsin and incubated at 37° C. for 3 days until plaques formed. The wells were fixed with 4% formaldehyde and stained with crystal violet, then dried. The wells are visible in FIG. 1.

As is visible in FIG. 1, the cell cultures treated with nitric oxide exposed virus (left hand side) exhibited significantly less influenza A virus as compared to the air control (right hand side) at each level of exposure (1, 2, and 3 hours). The cultured cells treated with NO-exposed virus illustrate that nitric oxide gas acts as an anti-viral agent. Additionally, the experiment provides the basis for using exposure to nitric oxide gas in the preparation of anti-viral vaccines.

### EXAMPLE 2

A special incubator with nitric oxide exposure capabilities was designed and built based on previously published work referenced above, “A direct nitric oxide gas delivery system for the bacterial and mammalian cell cultures.” This device enables the safe delivery of 160 ppm gNO within the level 3 biosafety containment laboratory at the British Columbia Centre for Disease Control. Additionally, a computer drive gas manifold was designed and built so that timing and dosing could be automated to increase the safety for the laboratory researchers.

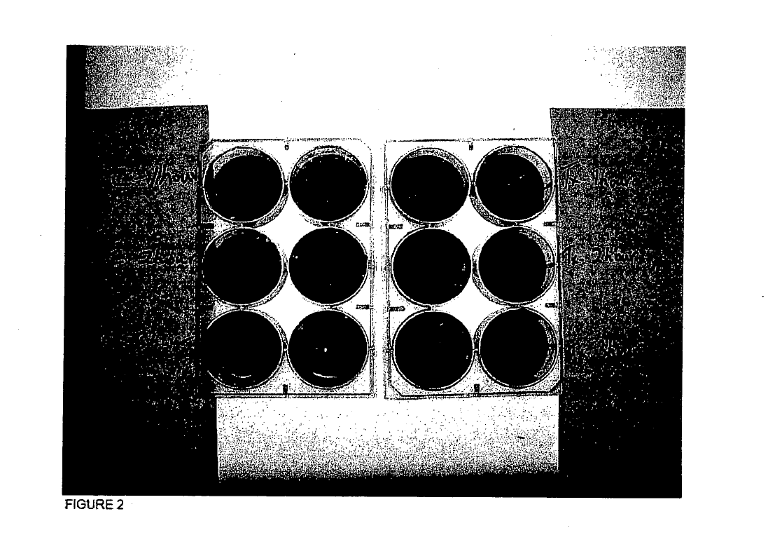

A surrogate strain of Influenza H3N2 was obtained from the laboratory stock of an academic virologist. The experiment was 1 mL of 6×10⁵ pfu placed in 3 wells of a 6 well plate and exposed for 1, 2 and 3 hours to either 160 ppm gNO (Tx) or room air (C or control). At each exposure time, the volume from each well (1 mL from 3 wells=3 mL) was extracted and frozen at −70° C. Madline-Darby canine kidney (MDCK) cells were grown in 6 well plates to a confluent monolayer. When cells were ready, 0.5 mL of each time point for treatment and control were inoculated into the cells and incubated on a shaker try for 1 hour at 37° C. The wells were then fixed with agar/trypsin and incubated at 37° C. for 3 days until plaques formed. The wells were then fixed with 4% formaldehyde and stained with crystal violet, the dried. These studies resulted in FIG. 2 showing that at each time point, the number of viruses capable of infecting a cell was reduced after being exposed to 160 ppm gNO to the room air control. In FIG. 2, the 6 wells on the left side are the control wells (room air); the 6 wells on right side are the treatment wells (160 ppm gNO). The top row of wells was exposed for 1 hour, the middle for 2 hours, and the bottom row for 3 hours. The wells of FIG. 2 illustrate that 160 ppm gNO significantly reduced the concentration of influenza A/H3N2.

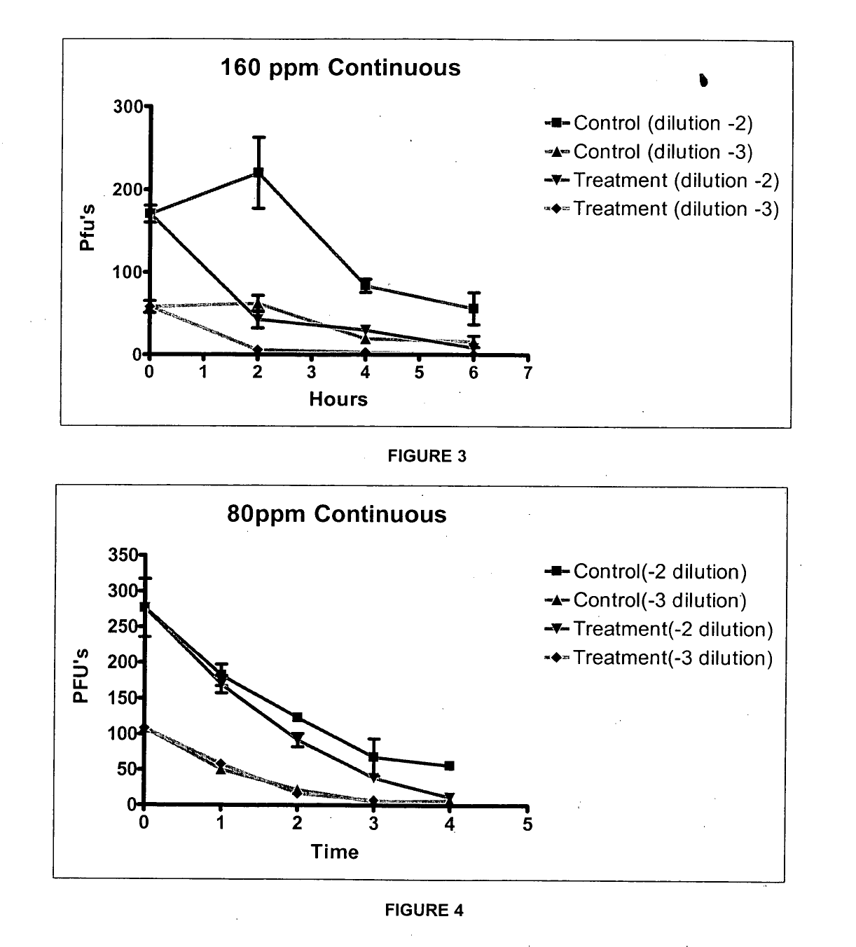

Additional studies were performed with influenza A/H3N2. Continuous exposure of gNO over 6 hours for two concentrations (dilutions) was plotted graphically in FIG. 3. FIG. 3 shows the results of continuous exposure of 160 gNO on influenza A/H3N2.

Using the same testing protocol, exposure of influenza A/H3N2 was exposed to 80 ppm gNO. The results are illustrated in FIG. 4, wherein wells were exposed continuously to gNO for 4 hours. This testing shows that 80 ppm gNO is not as effective as the 160 ppm concentration.

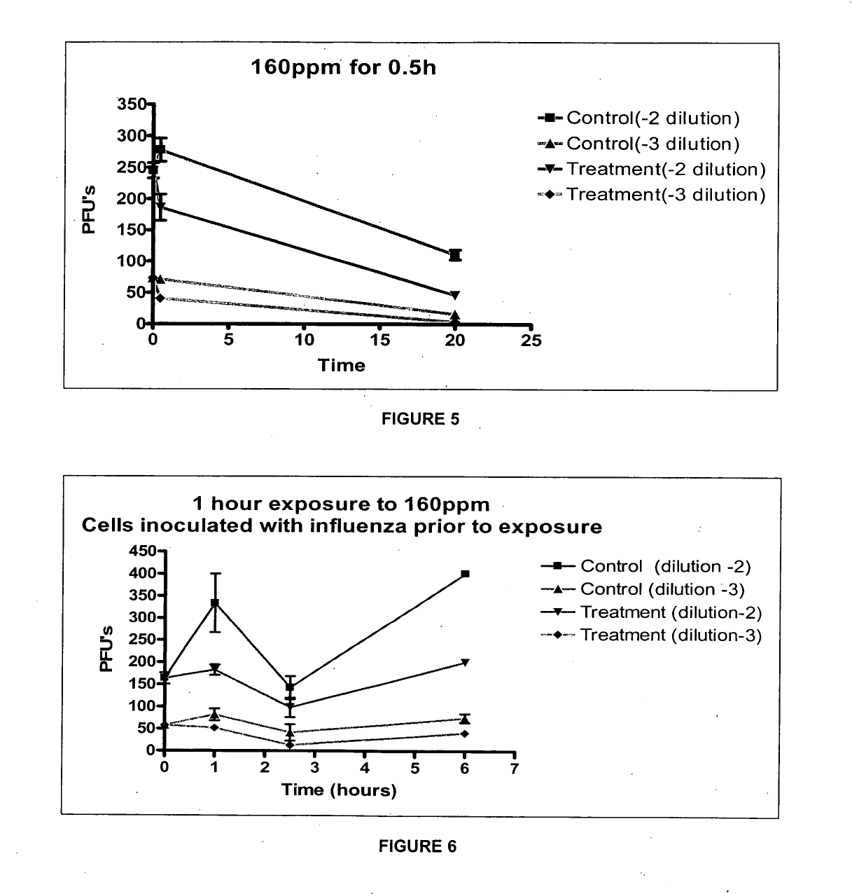

Next, a single exposure of 160 ppm gNO for 30 minutes was tested on influenza A/H3N2. The results are displayed in FIG. 5. This graph shows that just a single exposure for 30 minutes decreases concentration of influenza A/H3N2 over 20 hours (the well was sampled and plaque assay done to 20 hours after the 30 minute exposure).

Next, MDCK cells infected for 1 hour with influenza A/H3N2 were then exposed to 160 ppm gNO continuously for 6 hours. FIG. 6 is the graphical representation of influenza A/H3N2 infecting MDCK cells for 1 hours and then exposing to gNO 160 ppm continuously for 6 hours.

The next several experiments were conducted with influenza A/Victoria/H3N2.

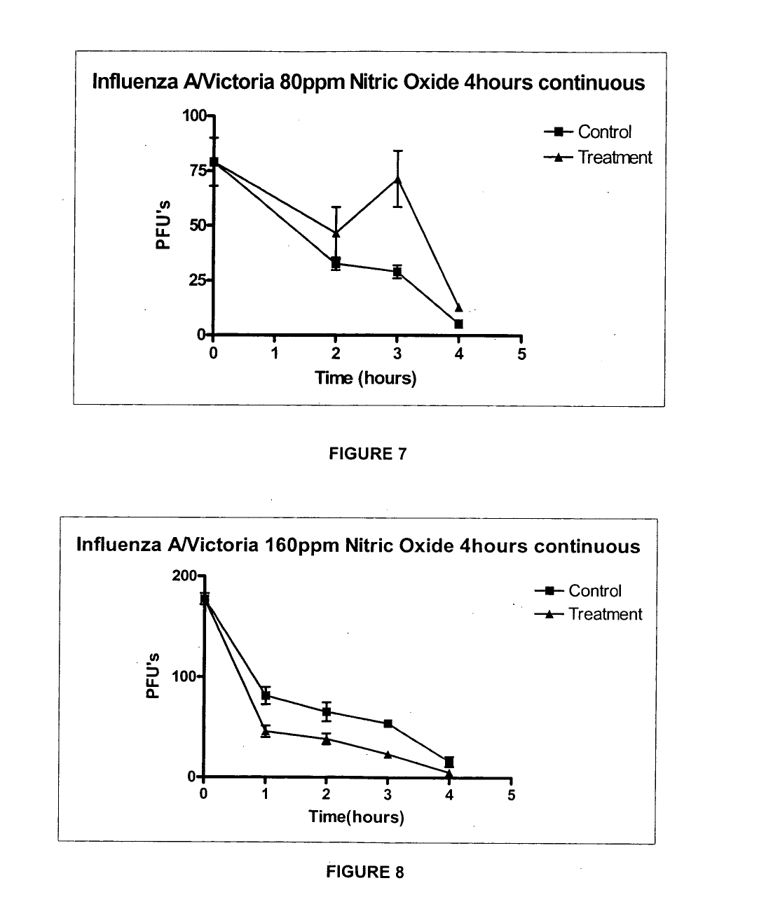

First, influenza A/Victoria/H3N2 was exposed to 80 ppm gNO continuously for 4 hours. These results are seen in FIG. 7. The 80 ppm gNO concentration was not effective against influenza A/Victoria/H3N2.

Influenza A/Victoria/H3N2 was next exposed to 160 ppm gNO for 4 continuous hours. These results are seen in FIG. 8. 160 ppm gNO is seen to be effective in reducing the concentration of influenza A/Victoria/H3N2.

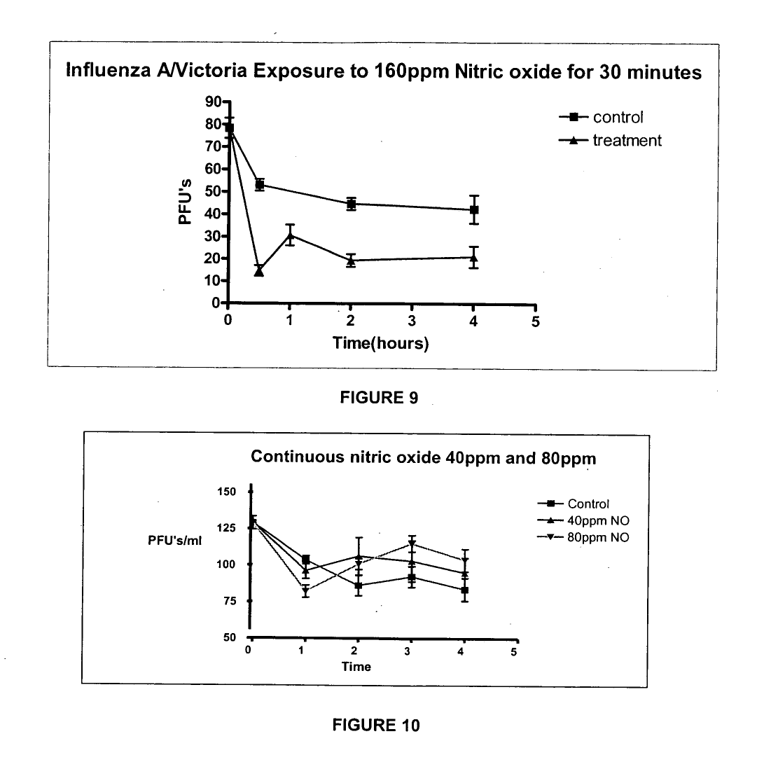

Next, influenza A/Victoria/H3N2 was exposed to 160 gNO for 30 minutes. The concentration of influenza A/Victoria/H3N2 was then tracked over 4 hours (sampled and plaque assay done to 4 hours). These results are shown in FIG. 9, which shown that a single exposure to 160 gNO for 30 minutes was effective in reducing concentration of influenza A/Victoria/H3N2.

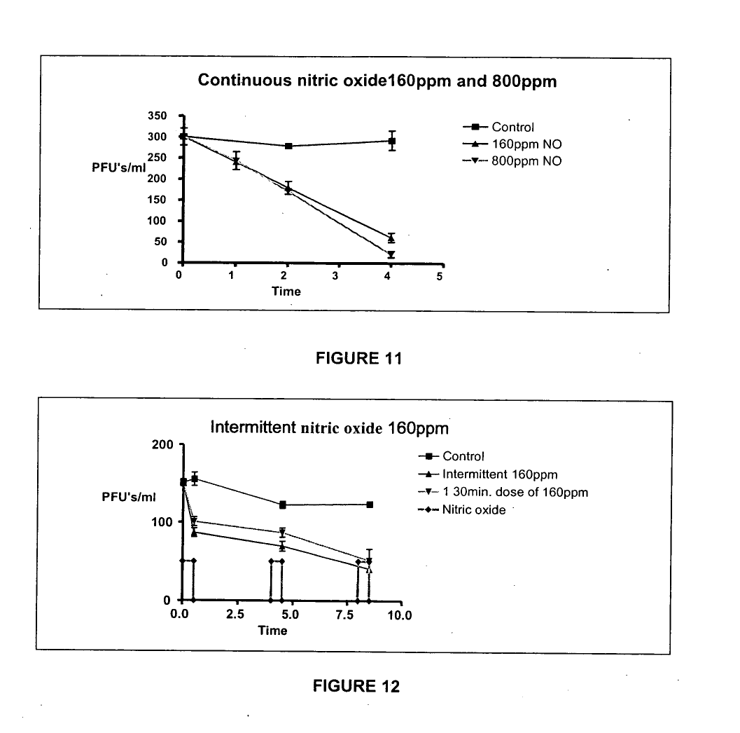

Experiments with influenza A/Victoria/H3N2 and exposure to gNO were repeated for several concentrations of gNO. These results are seen in FIGS. 10 and 11. There were 4 concentrations of gNO applied continuously over 4 hours to influenza A/Victoria/H3N2. In FIG. 10, concentrations of 40 ppm and 80 ppm were tested. While in FIG. 11, concentrations of 160 ppm and 800 ppm were tested. These show that a concentration of 160 ppm gNO and above is effective in reducing the infectivity of influenza A/Victoria/H3N2 compared to the controls and lower concentrations. However, a dosage of 160 ppm is probably safer than a dosage of 800 ppm.

In several patent applications, such as PCT/US2005/016427, filed on May 11, 2005, herein incorporated by reference in its entirety, intermittent dosages of gNO have been described. According to these patent applications, hypothetically, in order for nitric oxide gas to be effective as an inhaled drug for antimicrobial treatment, it may be delivered continuously for about 30 minutes at a time. Nitric oxide is inactivated by hemoglobin to form methemoglobin. The safe level of methemoglobin during inhaled nitric oxide therapy is less than 3%. The half life of methemoglobin is 1 hours in humans, thus nitric oxide gas could be delivered for 30 minutes every four hours without increasing the methemoglobin above safe levels. An intermittent study was devised to see if the viral infectivity was consistent with this administrative regimen. Thus, in FIG. 12, intermittent doses of 160 ppm gNO was compared to a control, continuous 160 ppm gNO exposure over 4 hours, and a single 30 minute 160 ppm gNO exposure. The intermittent dose means that gNO was delivered for 30 minutes once every hour for 4 hours. The single 30 minute exposure was handled as it was in previously described experiments, i.e., exposed and then sampled and plaque assay done to 4 hours. The intermittent dose was more effective than the single 30 minute exposure of 160 ppm gNO. Thus, the 160 ppm gNO delivered for 30 minutes every 4 hours was effective as an anti-infective agent against influenza A/Victoria/H3N2.

### EXAMPLE 3

Nitric oxide gas was tested against Highly Pathogenic Avian Influenza (HPAI). A strain of HPAI H7N3 was obtained. This strain was infected into confluent MDCK cell in a tissue culture flask where the virus propagated and released large quantities of virus into the supernatant. The supernatant was collected and centrifuged which then produced a quantity of virus which was then aliquoted into several freezer vials to be used as reference stock in these experiments. From the reference stock, serial dilutions of the virus were performed and then 1 mL of each dilution was placed in a 37 C incubator and the treated samples were exposed to 160 ppm gNO for 1, 2 and 3 hours within the treatment chamber, which was within a direct vented biosafety cabinet in a level 3 biocontainment lab (BCL3). At each point, the control and treated samples were inoculated onto confluent MDCK cells in 6 wells and the experiment proceeded as described above for Example 2 related to Influenza A.

The HPAI H7N3 stain that was exposed to gNO behaved in a similar manner to Influenza A. Nitric oxide gas at 160 ppm was able to reduce the infectivity of HPAI H7N3 after exposure of 3 continuous hours.

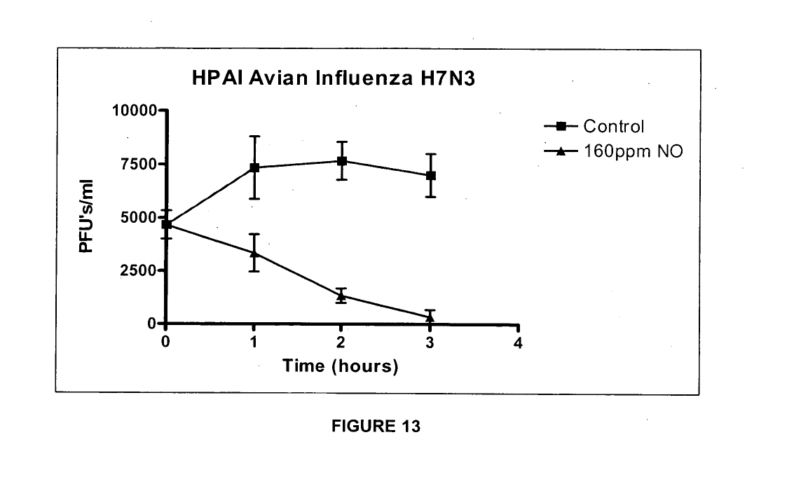

As seen in FIG. 13, 160 ppm gNO was applied to HPAI H7N3 continuously for 3 hours, resulting in a significant decrease in HPAI H7N3 infectivity. At zero (0) hours, the concentration of HPAI H7N3 is about 5000 PFU/mL, while after about 3 hours pf gNO exposure, the concentration has dropped to about 330 PFU/mL.

### EXAMPLE 4

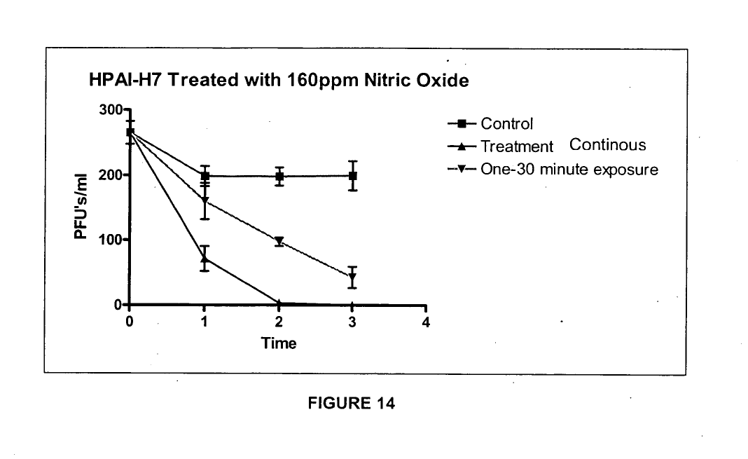

HPAI H7N3 was exposed to a single 30 minute exposure of 160 ppm gNO and to continuous exposure as shown in FIG. 14. 1 cryovial, 0.5 mL of 1×10⁵, HPAI H7N3 was obtained. From this sample, 100 μl was used to inoculate confluent MDCK cells to make freezer stock of the virus. The remainder of the sample was used to perform serial dilutions down to 10² and 10¹. These dilutions were then placed into wells for treatment. The controls were placed in a 37° C. incubator and the treatment samples were placed into the Treatment chamber at 160 ppm gNO. After 30 minutes one well was removed from the treatment chamber and placed in the incubator. Samples were obtained at 0, 1, 2 and 3 hours and the 0.5 mL of were inoculated onto confluent MDCK cell and were incubated for 1 hour. After 1 hour the inoculums were removed and the plates fixed with 2×MEM/agar and incubated for 44 hour. The agar was removed and the plates stained with crystal violet. The plaques were then photographed and counted.

Nitric oxide gas at 160 ppm was applied continuously over 2 hours to HPAI H7N3 as well as a single 30 minute exposure of gNO. See FIG. 14. The continuous exposure was comparatively better than the single treatment, but the single treatment still reduced the infectivity of the virus to under 50 PFU/mL after 3 hours.

### EXAMPLE 5

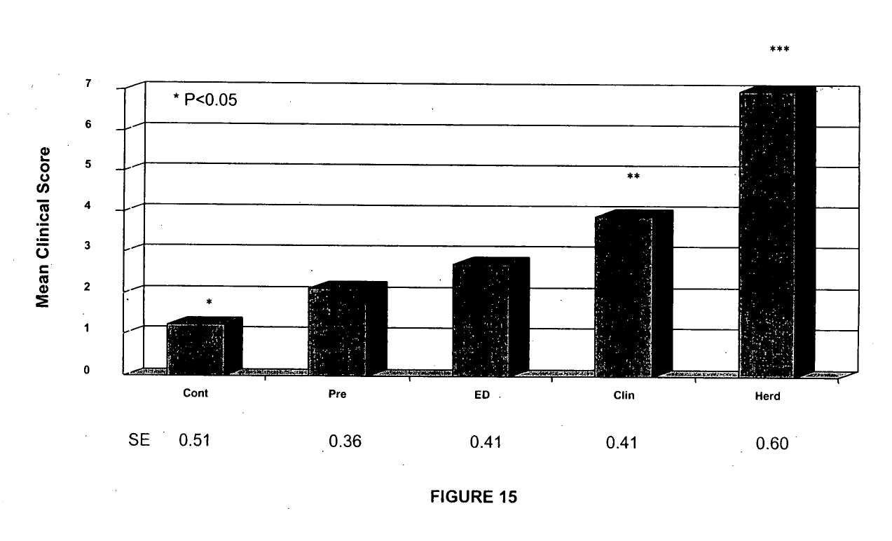

In vivo bovine study corresponding to FIGS. 15-18. Bovine Respiratory Disease (BRD) has a negative economic impact on the cattle industry. Some studies have shown that BRD accounts for 65-77% morbidity and 44-72% mortality in the beef industry. Further, there is dependence on prophylactic antibiotic use with resistant microbes emerging. Thus the need for an alternative treatment is present.

A natural inoculation pilot study using a Bovine Respiratory Virus (BRV) model was undertaken. Thirteen (13) infected naive calves were exposed for 52 hours to a commercial BRV positive induction herd. Calves were then pre-randomized into 1 of 4 groups:
1. Prophylactic (n=4), with NO treatment started immediately after heard exposure (Abbreviated “Pre” in FIGS. 15-18);
2. Early Detection (n=4), with NO treatment starting upon thermal infrared (IRT) detected signs of infections (Abbreviated “ED” in FIGS. 15-18). See e.g., “Early detection of BRD with Infrared orbital themography,” Schaefer et al. Can. J. An. Sci. 2004: 84:73, herein incorporated by reference in its entirety, wherein a mechanism using orbital infrared themography (IRT) is used to determine viral infection.
3. Clinical detection (n=3), with NO treatment initiated only upon evidence of industry standard clinical signs for BRD (Abbreviated “Clin” in FIGS. 15-18); and
4. Controls (n=2), naive calves were isolated and served as uninfected controls receiving a daily air placebo treatment (Abbreviated “Cont” in FIGS. 15-18).
5. The Induction Herd (labeled as “Herd” in FIGS. 15-18) was also monitored over the 4 days.

Once initiated, a single NO treatment was given each day for 4 consecutive days. Inhaled NO gas of 160 ppm was administered during the inspiratory phase via a nasal J-tube into each nare for 600 breaths (approximately) 20 minutes in duration. Methods of delivering inhaled gNO through a J-tube to equine have been described in U.S. Pat. No. 6,920,876, herein incorporated by reference in its entirety. Similar methods and devices were used to deliver the gNO to the bovine in this study. The presence of BRD related viruses were verified by relevant clinical signs. All calves had similar temperatures and clinical scores upon the initiation of the study.

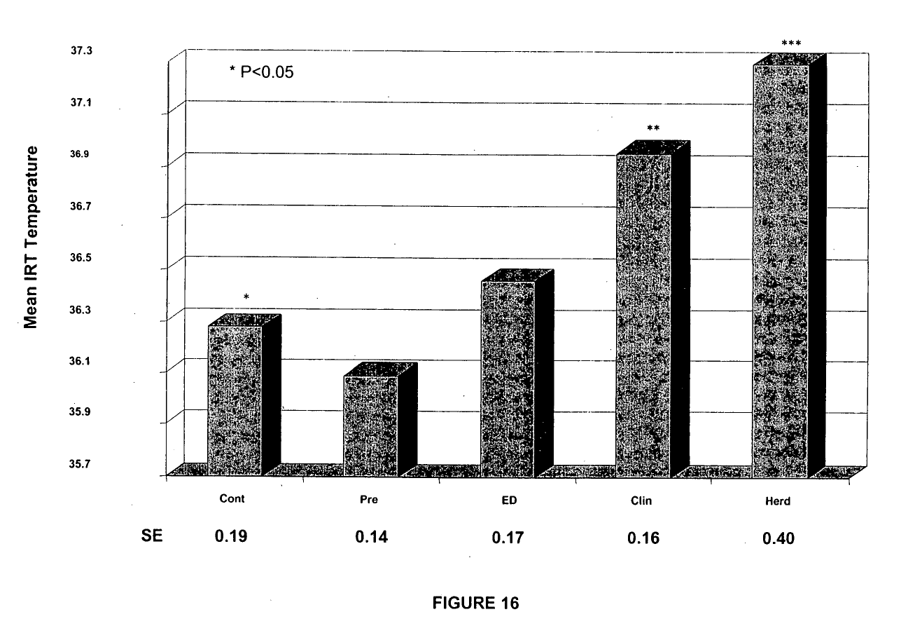

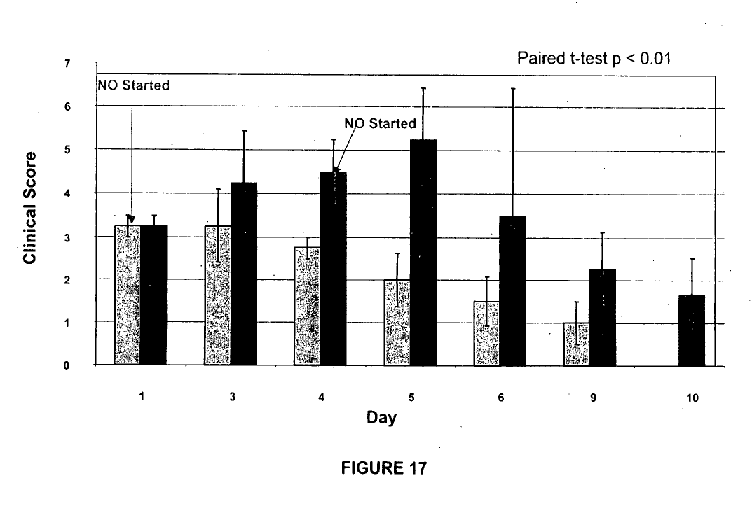

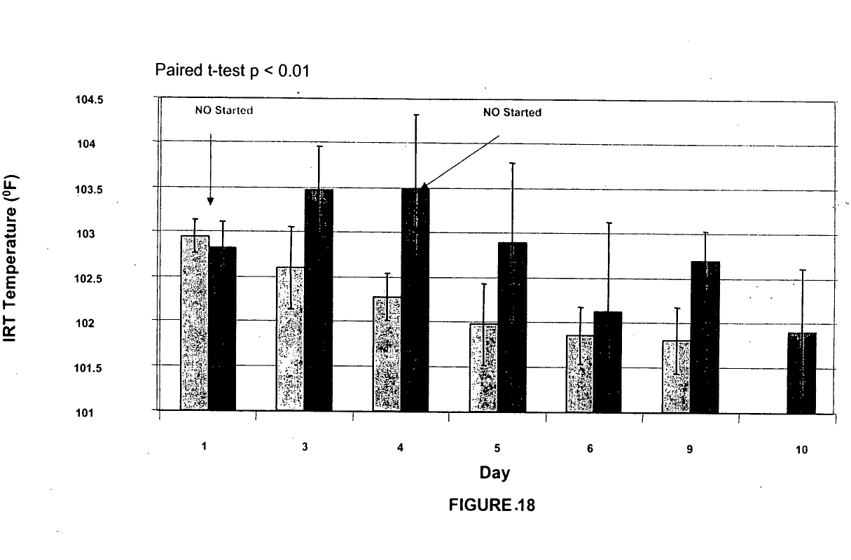

NO treated groups and the induction herd had positive serology for Para-influenza virus, Bovine Coronavirus, Respiratory Syncytial Virus, Bovine Virus Diarrhea and Infectious Bovine Rhinotracheitis. Prophylactic NO treatment was effective in preventing infection as seen in FIGS. 15-18. The clinical scores in FIGS. 15 and 17 are based on industry standard signs. In FIG. 15, it is shown that the lowest clinical score was for the control, then prophylactic, then early detection, the clinical, and then heard. The mean clinical scores represent the 10 day mean clinical infection score (industry standard signs). FIG. 16 shows Mean Temperatures across the groups, with the prophylactic group outperforming even the control group for lowest temperature of roughly 36.1° C. The Mean Temperatures were calculated over 10 days. FIG. 17 illustrates clinical scores for the prophylactic and early detection groups. 160 ppm of gNO was more effective in achieving low clinical scores for the prophylactic group than for the early detection group. In FIG. 18, the IRT Temperature is shown for the prophylactic and early detection groups. Again, 160 ppm of gNO was more effective in achieving low IRT temperatures for the prophylactic group than for the early detection group.

After 6 months, none of the NO treated animals demonstrated infection regardless of NO treatment dosing/timing. In comparison, the induction herd became ill and 13 out of 15 required antibiotic treatment within 6 months. Of these 13, 8 were treated directly for severe respiratory infections. When NO was given at the time of Early Detection (IRT) or at the point of Clinical Detection, the calves' temperatures and clinical scores rapidly resolved. The induction heard had a clinical score of 7.1 (standard error (SE) of 0.6) and also exhibit aberrant lab values. Paired t-test differences were highly significant (p<0.01) with similar scoring patterns. gNO of 160 ppm was delivered safely without incident.

### EXAMPLE 6

A similar in vivo bovine study as in Example 5 was conducted with one hundred and fifty-eight (158) calves. The calves were shipped into a commercial feedlot, wherein some of the animals at the feedlot were infected with BRV. The 152 calves were treated with either: (1) 4 minutes of approximately 160-260 ppm nitric oxide gas (30-60 breaths); or (2) conventional antibiotic, antiviral and immunization medications. 42 calves were treated with gNO, while 116 calves were treated with conventional antibiotics. Results showed that after 15 days, 9.5% (4/42) of those treated only with 4 minutes of nitric oxide gas exhibited clinical signs of infection (Bovine Respiratory Virus), whereas 13.8% (16/116) in the conventional treatment group exhibited clinical signs of infection (Bovine Respiratory Virus).

This test demonstrates that nitric oxide was almost twice as effective as an antiviral agent reducing infectivity as opposed to conventional vaccines, antiviral agents and antibiotics in this population. Further, these results suggest that as little as 400 breathes of 200 ppm nitric oxide gas make act as an immunological therapeutic vaccine.

### No Reduces Viral Infectivity

While not wishing to be bound by theory, it is believed that while viruses do not by themselves have thiol based detoxification pathways, they may still be inherently more susceptible to nitrosative stress. NO may inhibit viral ribonucleotide reductase, a necessary constituent enzyme of viral DNA synthesis and therefore inhibit viral replication. Nitric oxide may also inhibit the replication of viruses early during the replication cycle, involving the synthesis of vRNA and mRNA encoding viral proteins. With viruses also depending on host cells for detoxification of the body's defense pathways, the direct cytotoxic mechanisms of NO entering the host cells and the intracellular changes it produces, could also account for the viricidal effects through viral DNA decontamination. Thus, it is believed that the delivery of NO gas may also be effective against viruses.

While not wishing to be bound by theory, the Applicant believes that the NO molecule attacks the cysteine sites or nitrosylates the sulfur bonds in the hemaglutinin towers on the surface of the virus, such as the Influenza A virus. Specifically, the nitric oxide molecule may bind to the cysteine groups, thus altering the structure of hemagglutinin (HA) and/or neuraminidase (NA). Alternatively, it is believed that the NO molecule results in S-nitrosylation, altering the structure of HA and/or NA. Furthermore, the NO molecule may target and bond with the amines of the DNA and RNA of the virus, as explained above. Regardless of the mechanism, the Applicant shows that exposure of the virus to nitric oxide gas renders the virus non-infectious in human epithelial cells.

As a results of these experiments, it is theorized that NO binds to sites (epitopes) or alters the trimeric hemeglutinin structure by binding to sulphur and/or cysteine sites on the virus structure and prevents it from infecting the cell (attached to cell membrane). There are several reasons to support this theory. First, there appears to be no effect with lower doses of gNO (40 and 80 ppm). However, there is a significant effect at 160 ppm gNO. Increasing the does to 800 ppm had no real improved effect compared to 160 ppm. This observation suggests that there are only a certain number of sites that NO binds to and increasing NO dose beyond the effective number of binding sites is not more effective. Second, the intermittent dosage data suggests that even further that there are only a finite number of binding sites for NO. A single dose of 160 ppm seems to be as effective as three (3) thirty (30) minute doses of gNO. This suggests that the target sites on the virus, once bound with NO molecules, not only prevent fusing to the host cell membrane (reducing infectivity), but also allows the remaining viral structure with NO bound to it to act as an antigen in the host immune system. Finally, the bovine study of Example 5 shows that 4 single daily doses of about 20 minute inhalation sessions of 160 ppm reduces clinical symptoms of viral infections. Example 6 shows that treated with 4 minutes of approximately 160-260 ppm nitric oxide gas (30-60 breaths) reduces clinical symptoms of viral infections. Only 4 minutes of gNO exposure is almost twice as effective as conventional vaccines, antiviral agents and antibiotics.

Several researchers have documented the antiviral effects of the NO molecule produced chemically by NO donors. For example, cells infected with influenza virus A/Netherlands/18/94 were treated with NO, an experiment described in Rimmelzwaan, et. al., “Inhibition of Influenza Virus Replication by Nitric Oxide,” J. Virol. 1999; 73:8880-83, herein incorporated by reference in its entirety. Results show the effectiveness of NO as a preventive therapy to viral agents. Additionally, a study by Sanders, et. al. demonstrates the effectiveness of naturally produced NO by the body as an antiviral agent, particularly against human rhinovirus. See Sanders, et. al., “Role of Nasal Nitric Oxide in the Resolution of Experimental Rhinovirus Infection,” J. Allergy Clin, Immunol. 2004 April; 113(4):697-702, herein incorporated by reference in its entirety.

As such, exposure of the virus to both NO gas and the NO molecule results in an activated or attenuated virus, which may be used in formulating vaccines. Treatments according to the present invention thus include gNO exposure in combination with one or more of a NO releasing compound, a vaccine adjuvant, and an anti-viral adjuvant. Vaccine adjuvants and anti-viral adjuvants include know traditional anti-biotic treatments for viral infections and other known viral inoculations.

NO gas may be used to inactivate or attenuate any virus strand, such as, but not limited to Influenza A and B, Avian Flu viruses, such as the H5N1 variant and others, SARS (Corona viruses) viruses, HIV, respiratory syncytial, and Rhinoviruses. The Applicant understands that the Avian Flu virus may mutate from its current variant into other strains, known as an antigenic shift and antigenic drift. It is specifically contemplated that the present methods, treatments, and vaccines be suitable for the current and future variants of Avian Flu viruses. Viral infections affect both animals and plants. As such, treatments discussed herein may be used for humans, animals, mammals, such as cattle, birds, fish, and plants, such as tobacco.

The delivery of gNO to the virus may be accomplished through any known delivery method, particularly through continuous or intermittent exposure to gNO for times sufficient to inactivate or attenuate the virus. Any known delivery devices, systems and methods may be used to expose to the virus to gNO. One exposure mechanism is the one described in the example above, the exposure chamber. Other gNO delivery systems have been described in PCT Application No. PCT/US2005/016427, herein incorporated by reference. In another example, devices known to deliver NO gas topically to a surface of the body such as a skin or eye, a surface of an organ such heart, stomach, etc., a bathing unit as described in U.S. Pat. No. 6,432,077, may be used to delivery NO to the virus in vitro. U.S. Pat. No. 6,432,077 is hereby incorporated by reference as if fully set forth herein. Another example of a delivery is an interface to a dialysis circuit or extracorporeal circuitry wherein the NO gas is delivered directly to the blood or body fluids so as to expose the blood or body fluids to NO gas. Such delivery interface is described, for example, in U.S. patent application Ser. Nos. 10/658,665, filed on Sep. 9, 2003 and 11/445,965, filed on Jun. 5, 2006, which are hereby incorporated by reference in their entirety. It should be understood that the types of delivery methods should not be limiting.

Accordingly, concentrations greater than 100 ppm nitric oxide and, more preferably, about or greater than 160 ppm nitric oxide may be applied to the virus. Preferably, the concentration of nitric oxide in the nitric oxide containing gas that contacts the virus is about 120 ppm to about 400 ppm, more preferably, about 160 ppm to about 220 ppm.

From the Examples, it is seen that gNO exposure is effective against several viruses and as a treatment in a patient with a viral infection. Thus, the data shows that gNO is effective for treating viral infection and as an agent that inactivates, attenuates, reduces infectivity or eradicates the virus directly. Thus, the methods claimed herein are directed to methods of treating patients, methods of inactivating a virus, methods of producing a treated virus for use in a vaccine, and methods of producing a vaccine. Additionally, vaccines are claimed herein.

In addition to gNO, nitric oxide releasing compounds are also effective in methods of treating patients, methods of inactivating a virus, methods of producing a treated virus for use in a vaccine, and methods of producing a vaccine. Thus, a patient may be treated with or a virus may be exposed to a therapeutically effective amount of an NO-releasing, NO-donor, or NO-upregulator compound. For simplicity, NO-releasing, NO-donor and NO-upregulators will be referred to only as “NO-releasing compounds.” Known NO-releasing compounds useful in the methods and devices of the invention include, but are not limited to: nitroso or nitrosyl compounds characterized by an —NO moiety that is spontaneously released or otherwise transferred from the compound under physiological conditions(e.g. S-nitroso-N-acetylpenicillamine, S-nitroso-L-cysteine, nitrosoguanidine, S-nitrosothiol, and others described in WO 92/17445 and U.S. Pat. No. 5,427,797 (herein incorporated by reference)). In addition, other NO-releasing compounds include compounds in which NO is a ligand on a transition metal complex, and as such is readily released or transferred from the compound under physiological conditions (e.g. nitroprusside, NO-ferredoxin, NO-heme complex) and nitrogen-containing compounds which are metabolized by enzymes endogenous to the respiratory and/or vascular system to produce the NO radical (e.g. arginine, glyceryl trinitrate, isoamyl nitrite, inorganic nitrite, azide and hydroxylamine). More NO-releasing compounds are polyethyleneimine (PEI)-based polymers exposed to NO gas; molsidomine; nitrate esters; sodium nitrite; iso-sorbide didinitrate; penta erythritol tetranitrate; nitroimidazoles; complexes of nitric oxide and polyamines; anionic diazeniumdiolates (NONOnates) (including those disclosed in U.S. Pat. Nos. 4,954,526 and 5,155,137) and the NO releasing compounds disclosed in U.S. Pat. No. 5,840,759 and PCT WO 95/09612. Examples of NONOate compounds include diethylamine/NONO, diethylenetriamine/NONO, and methylaminohexylmethylamine/NONO (illustrated in Hanson et al., Nitric Oxide, Biochemistry, Molecular Biology, and Therapeutic Implications, Ignarro and Murad, Ed., Academic Press, New York (1995)). An NO-releasing compound, donor or upregulator can be provided in powder form or as a liquid (e.g., by mixing the compound with a biologically-compatible excipient).

The NO-releasing compound may be administered to a patient alone or in conjunction with NO gas, CO gas, a carrier gas or another NO-releasing compound. When more than one compound is administered to the patient, the compounds can be mixed together, or they can be administered to the patient sequentially. Any one, or a combination, of the following routes of administration can be used to administer the NO-releasing compound(s) to the patient: intravenous injection, intraarterial injection, transcutaneous delivery, oral delivery, and inhalation (e.g., of a gas, powder or liquid).

The NO-releasing compound selected for use in the method of the invention may be administered as a powder (i.e., a finely divided solid, either provided pure or as a mixture with a biologically-compatible carrier powder, or with one or more additional therapeutic compounds) or as a liquid (i.e., dissolved or suspended in a biologically-compatible liquid carrier, optionally mixed with one or more additional therapeutic compounds), and can conveniently be inhaled in aerosolized form (preferably including particles or droplets having a diameter of less than 10 μm). Carrier liquids and powders that are suitable for inhalation are commonly used in traditional asthma inhalation therapeutics, and thus are well known to those who develop such therapeutics. The optimal dosage range can be determined by routine procedures by a pharmacologist of ordinary skill in the art. For example, a useful dosage level for SNAP would be from 1 to 500 μmoles (preferably 1-200 μmoles) per inhaled dose, with the number of inhalations necessary varying with the needs of the patient.

When an NO-releasing compound is inhaled in solid or liquid form, the particles or droplets are deposited throughout the respiratory system, with larger particles or droplets tending to be deposited near the point of entry (i.e., in the mouth or nose) and smaller particles or droplets being carried progressively further into the respiratory system before being deposited in the trachea, bronchi, and finally the alveoli. (See, e.g., Hounam & Morgan, “Particle Deposition”, Ch. 5 in Respiratory Defense Mechanisms, Part 1, Marcel Dekker, Inc., NY; ed. Brain et al., 1977; p. 125.) A particle/droplet diameter of 10 .mu.m or less is recommended for use in the method of the invention. Determination of the preferred carrier (if any), propellant (which may include NO diluted in an inert gas such as N.sub.2), design of the inhaler, and formulation of the NO-releasing compound in its carrier are well within the abilities of those of ordinary skill in the art of devising routine asthma inhalation therapies. The portable inhaler could contain an NO-releasing compound either mixed in dry form with a propellant or held in a chamber separate from the propellant, or mixed with a liquid carrier capable of being nebulized to an appropriate droplet size, or in any other configuration known to those skilled in portable inhaler technology. A few of the several types of inhaler designs that have been developed to date are discussed in, for example, U.S. Pat. Nos. 4,667,668; 4,592,348; 4,534,343; and 4,852,561, each of which patents is herein incorporated by reference. Other inhaler designs are described in the Physicians' Desk Reference, 45th Edition, Edward R. Barnhart, Publisher (1991). Each of these and other aerosol-type inhalers can be adapted to accommodate the delivery of NO-releasing compounds. Also useful for delivering an NO-releasing compound formulated in dry powder form is a non-aerosol-type inhaler device such as that developed by Allen & Hanburys, Research Triangle Park, North Carolina.

In addition to the production and use of vaccines, the above results demonstrate that the use of inhaled NO gas in combination with a traditional vaccine, NO releasing compound, antiviral agent or other adjuvant treatments for viral diseases will increase the effectiveness of the treatment. As such, NO gas alone or the exposed or treated virus may be used as a vaccine adjuvant, or an agent added to a vaccine to increase or aid its effect. One or more of nitric oxide gas, a NO releasing compound, a vaccine adjuvant, and an anti-viral adjuvant may be used to treat patients or directly inactivate a virus. Thus, the vaccines described herein may be further formulated with one or more of another vaccine, an anti-viral agent, a vaccine adjuvant, an anti-viral adjuvant, nitric oxide, and a nitric oxide releasing compound. Further, the methods described herein may further include administering one or more of a vaccine, an anti-viral agent, a vaccine adjuvant, an anti-viral adjuvant, nitric oxide, and a nitric oxide releasing compound.

Vaccines created from treated virus strains may be formulated into any effective dosage and duration. Vaccines may be delivered by injection, orally, intranasally, through inhalation, endotracheal tubes during mechanical ventilation, and through other known means. For example, delivery by inhalation or to the respiratory airway can be made to spontaneously breathing mammals or those managed with mechanical ventilation. With respect to spontaneously breathing mammals, delivery can be achieved via many gas delivery systems such as masks or nasal cannulas.

### Vaccines

The virus that has been exposed to nitric oxide gas is an inactivated or attenuated virus. In addition to the nitric oxide exposure, the virus may be treated with other known methods of inactivating or attenuating virus strains, such as those described in U.S. Pat. No. 6,344,354. Thus, the exposed virus may be used in any known inactivated or attenuated vaccine formulation and delivery.

The virus can thus be attenuated or inactivated, formulated and administered, according to known methods, as a vaccine to induce an immune response in a mammal or other living plant or animal. Methods are well-known in the art for determining whether such attenuated or inactivated vaccines have maintained similar antigenicity to that of the clinical isolate or high growth strain derived therefrom. Such known methods include the use of antisera or antibodies to eliminate viruses expressing antigenic determinants of the donor virus; chemical selection (e.g, amantadine or rimantidine); HA and NA activity and inhibition; and DNA screening (such as probe hybridization or PCR) to confirm that donor genes encoding the antigenic determinants (e.g., HA or NA genes) are not present in the attenuated viruses. See, e.g., Robertson et al., Giornale di Igiene e Medicina Preventiva 29:4-58 (1988); Kilbourne, Bull. M2 World Health Org. 41:643-645 (1969); Aymard-Henry et al., Bull. World Health Org. 481:199-202 (1973); Mahy et al., J. Biol. Stand. 5:237-247 (1977); Barrett et al., Virology: A Practical Approach, Oxford IRL Press, Oxford, pp. 119-150 (1985); Robertson et al., Biologicals 20:213-220 (1992).

### Pharmaceutical Compositions

A pharmaceutically acceptable carrier or diluent which may be formulated with a vaccine or provided in connection with a gNO treatment include one or more of nitric oxide gas, a NO releasing compound, and an adjuvant viral treatment compound.

Pharmaceutical compositions of the present invention, suitable for inoculation or for parenteral or oral administration, comprise attenuated or inactivated mammalian influenza viruses, optionally further comprising sterile aqueous or non-aqueous solutions, suspensions, and emulsions. The composition can further comprise auxiliary agents or excipients, as known in the art. See, e.g, Berkow et al., eds., The Merck Manual, 15th edition, Merck and Co., Rahway, N.J. (1987); Goodman et al., eds., Goodman and Gilman's The Pharmacological Basis of Therapeutics, 8th edition, Pergamon Press, Inc., Elmsford, N.Y. (1990); Avery's Drug Treatment: Principles and Practice of Clinical Pharmacology and Therapeutics, 3rd edition, ADIS Press, LTD., Williams and Wilkins, Baltimore, Md. (1987); Osol, A., ed., Remington's Pharmaceutical Sciences, Mack Publishing Co, Easton, Pa. pp. 1324-1341 (1980); Katzung, ed. Basic and Clinical Pharmacology, Fifth Edition, Appleton and Lange, Norwalk, Conn. (1992), which references and references cited therein, are entirely incorporated herein by reference as they show the state of the art.

A virus vaccine composition of the present invention can comprise from about 10²-10⁹ plaque forming units (PFU)/ml, or any range or value therein, where the virus is attenuated. A vaccine composition comprising an inactivated virus can comprise an amount of virus corresponding to about 0.1 to 200 μg of hemagglutinin protein/ml, or any range or value therein.

Preparations for parenteral administration include sterile aqueous or non-aqueous solutions, suspensions, and/or emulsions, which may contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents. See, e.g., Berkow, infra, Goodman, infra, Avery's, infra, Osol, infra and Katzung, infra, which are entirely incorporated herein by reference, included all references cited therein.

When a vaccine composition of the present invention is used for administration to an individual, it can further comprise salts, buffers, adjuvants, or other substances which are desirable for improving the efficacy of the composition. Adjuvants are substances that can be used to augment a specific immune response. Normally, the adjuvant and the composition are mixed prior to presentation to the immune system, or presented separately, but into the same site of the mammal being immunized. Examples of materials suitable for use in vaccine compositions are provided in Osol, A., ed., Remington's Pharmaceutical Sciences, Mack Publishing Co, Easton, Pa. (1980), pp. 1324-1341, which reference is entirely incorporated herein by reference.

Heterogeneity in the vaccine may be provided by mixing replicated influenza viruses for at least two mammalian influenza virus strains, such as 2-50 strains or any range or value therein. Influenza A or B virus strains having a modern antigenic composition are preferred. According to the present invention, vaccines can be provided for variations in a single strain of an influenza virus or for more than one strain of influenza viruses, using techniques known in the art.

A pharmaceutical composition according to the present invention may further or additionally comprise at least one viral chemotherapeutic compound, including, but not limited to, gamma globulin, amantadine, guanidine, hydroxybenzimidazole, interferon-α, interferon-β, interferon-γ, thiosemicarbarzones, methisazone, rifampin, ribavirin, a pyrimidine analog, a purine analog, foscarnet, phosphonoacetic acid, acyclovir, dideoxynucleosides, a protease inhibitor, or ganciclovir. See, e.g., Katzung, infra, and the references cited therein on pages 798-800 and 680-681, respectively, which references are herein entirely incorporated by reference.

The vaccine can also contain variable but small quantities of endotoxin, free formaldehyde, and preservative, which have been found safe and not contributing to the reactogenicity of the vaccines for humans.

### Pharmaceutical Purposes

The administration of the vaccine composition (or the antisera that it elicits) may be for either a “prophylactic” or “therapeutic” purpose. When provided prophylactically, the compositions are provided before any symptom of viral infection becomes manifest. The prophylactic administration of the composition serves to prevent or attenuate any subsequent infection. When provided therapeutically, the attenuated or inactivated viral vaccine is provided upon the detection of a symptom of actual infection. The therapeutic administration of the compound(s) serves to attenuate any actual infection. See, e.g, Berkow, infra, Goodman, infra, Avery, infra and Katzung, infra, which are entirely incorporated herein by reference, including all references cited therein.

An attenuated or inactivated vaccine composition of the present invention may thus be provided either before the onset of infection (so as to prevent or attenuate an anticipated infection) or after the initiation of an actual infection.

A composition is said to be “pharmacologically acceptable” if its administration can be tolerated by a recipient patient. Such an agent is said to be administered in a “therapeutically effective amount” if the amount administered is physiologically significant. A vaccine or composition of the present invention is physiologically significant if its presence results in a detectable change in the physiology of a recipient patient that enhances at least one primary or secondary humoral or cellular immune response against at least one strain of an infectious virus, such as an influenza or Avian flu virus.

The “protection” or “treatment” provided need not be absolute, i.e., the viral infection need not be totally prevented or eradicated, if there is a statistically significant improvement compared with a control population or set of patients. Protection may be limited to mitigating the severity or rapidity of onset of symptoms of the virus infection.

### Pharmaceutical Administration

A vaccine of the present invention may confer resistance to one or more virus, such as influenza strains by either passive immunization or active immunization. In active immunization, an inactivated or attenuated live vaccine composition is administered prophylactically, according to a method of the present invention. In another embodiment as passive immunization, the vaccine is provided to a host (i.e. a mammal), and the elicited antisera is recovered and administered to a recipient suspected of having an infection caused by at least one influenza virus strain.

The present invention thus includes methods for preventing or attenuating infection by at least one influenza virus strain. As used herein, a vaccine is said to prevent or attenuate a disease if its administration results either in the total or partial attenuation (i.e., suppression) of a symptom or condition of the disease, or in the total or partial immunity of the individual to the disease.

At least one inactivated or attenuated influenza virus, or composition thereof, of the present invention may be administered by any means that achieve the intended purpose, using a pharmaceutical composition as previously described.

For example, administration of such a composition may be by various parenteral routes such as subcutaneous, intravenous, intradermal, intramuscular, intraperitoneal, intranasal, oral or transdermal routes. Parenteral administration can be by bolus injection or by gradual perfusion over time. A preferred mode of using a pharmaceutical composition of the present invention is by intramuscular or subcutaneous application. See, e.g., Berkow, infra, Goodman, infra, Avery, infra and Katzung, infra, which are entirely incorporated herein by reference, including all references cited therein.

A typical regimen for preventing, suppressing, or treating an influenza virus related pathology, comprises administration of an effective amount of a vaccine composition as described herein, administered as a single treatment, or repeated as enhancing or booster dosages, over a period up to and including between one week and about 24 months, or any range or value therein. For example, the attenuated influenza virus may be packaged in a single-use syringe or aerosolized for delivery into the nostrils wherein the solution in the syringe or aerosol also contains nitric oxide gas or gas producing compounds.

According to the present invention, an “effective amount” of a vaccine composition is one that is sufficient to achieve a desired biological effect. It is understood that the effective dosage will be dependent upon the age, sex, health, and weight of the recipient, kind of concurrent treatment, if any, frequency of treatment, and the nature of the effect wanted. The ranges of effective doses provided below are not intended to limit the invention and represent preferred dose ranges. However, the most preferred dosage will be tailored to the individual subject, as is understood and determinable by one of skill in the art, without undue experimentation. See, e.g., Berkow et al., eds., The Merck Manual, 16th edition, Merck and Co., Rahway, N.J., 1992; Goodman et al., eds., Goodman and Gilman's The Pharmacological Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, Mass. (1985); and Katsung, infra, which references and references cited therein, are entirely incorporated herein by reference.

The dosage of an attenuated virus vaccine for a mammalian (e.g., human) adult can be from about 10³-10⁷ plaque forming units (PFU)/kg, or any range or value therein. The dose of inactivated vaccine can range from about 1 to 50 μg of hemagglutinin protein. However, the dosage should be a safe and effective amount as determined by conventional methods, using existing vaccines as a starting point.

The dosage of immunoreactive HA in each dose of replicated virus vaccine can be standardized to contain a suitable amount, e.g., 1-50 μg or any range or value therein, or the amount recommended by the U.S. Public Health Service (PHS), which is usually 15 μg, per component for older children ≧3 years of age, and 7.5 μg per component for children <3 years of age. The quantity of NA can also be standardized, however this glycoprotein can be labile during the process of purification and storage (Kendal et al., Infect. Immun. 29:966-971 (1980); Kerr et al., Lancet 1:291-295 (1975)). Each 0.5-ml dose of vaccine preferably contains approximately 1-50 billion virus particles, and preferably 10 billion particles.

While embodiments of the present invention have been shown and described, various modifications may be made without departing from the scope of the invention. The invention, therefore, should not be limited, except to the following claims, and their equivalents.

1. A method of inactivating or attenuating a virus comprising:
identifying a virus; and
exposing the virus to nitric oxide gas for a period of time and at a concentration sufficient to inactivate or attenuate the virus.

2. The method of claim 1, wherein the exposure step is in vivo.

3. The method of claim 2, wherein the exposing step is accomplished through inhalation of the nitric oxide gas into the respiratory tract of a patient.

4. The method of claim 1, wherein the exposure step is in vitro.

5. The method of claim 1, wherein the period of time is at least about 100-400 breaths.

6. The method of claim 5, wherein the period of time is from about 5-10 minutes to about 30 minutes every 4 hours.

7. The method of claim 1, wherein the nitric oxide gas has a concentration of about 120 ppm to about 400 ppm nitric oxide.

8. The method of claim 7, wherein the nitric oxide gas has a concentration of about 160 ppm.

9. The method of claim 1, wherein the virus is selected from Influenza A, Influenza B, Avian Flu viruses, SARS (Corona) viruses, respiratory syncytial viruses, para-influenza viruses, Bovine Virus Diarrhea, HIV, and Rhinoviruses.

10. The method of claim 9, wherein the virus is an Avian Flu virus.

11. The method of claim 1, further comprising the step of formulating the inactivated or attenuated virus with one or more of a vaccine, an anti-viral agent, a vaccine adjuvant, an anti-viral adjuvant, nitric oxide, and a nitric oxide releasing compound.

12. A method of producing a treated virus for use as a vaccine or anti-viral agent comprising:
identifying a virus; and
exposing the virus to nitric oxide gas for a period of time and at a concentration sufficient to inactivate or attenuate the virus.

13. The method of claim 12, wherein the exposure step is in vivo.

14. The method of claim 13, wherein the exposing step is accomplished through inhalation of the nitric oxide gas into the respiratory tract of a patient.

15. The method of claim 12, wherein the exposure step is in vitro.

16. The method of claim 12, wherein the period of time is at least 100-400 breaths.

17. The method of claim 16, wherein the period of time is from about 5-10 minutes to about 30 minutes every 4 hours.

18. The method of claim 12, wherein the nitric oxide gas has a concentration, of about 120 ppm to about 400 ppm nitric oxide.

19. The method of claim 18, wherein the nitric oxide gas has a concentration of about 160 ppm.

20. The method of claim 12, wherein the virus is selected from Influenza A, Influenza B, Avian Flu viruses, SARS (Corona) viruses, respiratory syncytial viruses, para-influenza viruses, Bovine Virus Diarrhea, HIV, and Rhinoviruses.

21. The method of claim 20, wherein the virus is an Avian Flu virus.

22. The method of claim 12, further comprising the step of formulating the inactivated or attenuated virus with one or more of a vaccine, an anti-viral agent, a vaccine adjuvant, an anti-viral adjuvant, nitric oxide, and a nitric oxide releasing compound.

23. A method of producing a vaccine comprising:
identifying a virus;
exposing the virus to nitric oxide gas for a period of time and at a concentration sufficient to inactivate or attenuate the virus;
adding the exposed virus to patient cells;
growing the infected and exposed patient cells; and
formulating the resulting grown cells into a vaccine.

24. The method of claim 23, wherein the period of time is at least about 20 minutes of continuous exposure.

25. The method of claim 23, wherein the nitric oxide gas has a concentration of about 120 ppm to about 400 ppm nitric oxide.

26. The method of claim 23, wherein the virus is selected from Influenza A, Influenza B, Avian Flu viruses, SARS (Corona) viruses, respiratory syncytial viruses, para-influenza viruses, Bovine Virus Diarrhea, HIV, and Rhinoviruses.

27. The method of claim 26, wherein the virus is an Avian Flu virus.

28. The method of claim 23, further comprising the step of formulating the inactivated or attenuated virus with one or more of a vaccine, an anti-viral agent, a vaccine adjuvant, an anti-viral adjuvant, nitric oxide, and a nitric oxide releasing compound.

29. A method of producing a vaccine comprising:
identifying a virus;
adding the virus to cultured patient cells;
exposing the infected cultured patient cells to nitric oxide gas for a period of time and at a concentration sufficient to inactivate or attenuate the virus;
growing the patient cells; and
formulating the resulting grown cells into a vaccine.

30. The method of claim 29, wherein the period of time is at least about 20 minutes of continuous exposure.

31. The method of claim 29, wherein the nitric oxide gas has a concentration of about 120 ppm to about 400 ppm nitric oxide.

32. The method of claim 29, wherein the virus is selected from Influenza A, Influenza B, Avian Flu viruses, SARS (Corona) viruses, respiratory syncytial viruses, para-influenza viruses, Bovine Virus Diarrhea, HIV, and Rhinoviruses.

33. The method of claim 32, wherein the virus is an Avian Flu virus.

34. The method of claim 29, further comprising the step of formulating the inactivated or attenuated virus with one or more of a vaccine, an anti-viral agent, a vaccine adjuvant, an anti-viral adjuvant, nitric oxide, and a nitric oxide releasing compound.

35. A vaccine made by any of the methods of claim 23-34.

36. A vaccine comprising:
a pharmaceutically acceptable carrier or diluent; and
an exposed virus that is inactivated or attenuated, wherein the exposed virus is obtained by:
identifying a virus; and
exposing the virus to nitric oxide gas for a period of time and at a concentration sufficient to inactivate or attenuate the virus.

37. The vaccine of claim 36, wherein the period of time is at least about 20 minutes of continuous exposure.

38. The vaccine of claim 36, wherein the nitric oxide gas has a concentration of about 120 ppm to about 400 ppm nitric oxide.

39. The vaccine of claim 36, wherein the virus is selected from Influenza A, Influenza B, Avian Flu viruses, SARS (Corona) viruses, respiratory syncytial viruses, para-influenza viruses, Bovine Virus Diarrhea, HIV, and Rhinoviruses.

40. The vaccine of claim 39, wherein the virus is an Avian Flu virus.

41. The vaccine of claim 36, wherein the vaccine further comprises one or more of another vaccine, an anti-viral agent, a vaccine adjuvant, an anti-viral adjuvant, nitric oxide, and a nitric oxide releasing compound.

42. A method of treating a patient comprising:
providing a vaccine to a patient, wherein the vaccine comprises:
an exposed virus that is inactivated or attenuated, wherein the exposed virus is obtained by:
identifying a virus; and
exposing the virus to nitric oxide gas for a period of time and at a concentration sufficient to inactivate or attenuate the virus.

43. The method of claim 42, wherein the period of time is at least about 20 minutes of continuous exposure.

44. The method of claim 42, wherein the nitric oxide gas has a concentration of about 120 ppm to about 400 ppm nitric oxide.

45. The method of claim 42, wherein the virus is selected from Influenza A, Influenza B, Avian Flu viruses, SARS (Corona) viruses, respiratory syncytial viruses, para-influenza viruses, Bovine Virus Diarrhea, HIV, and Rhinoviruses.

46. The method of claim 45, wherein the virus is an Avian Flu virus.

47. The method of claim 42, wherein the patient has a viral infection.

48. The method of claim 42, further comprising administering to the patient one or more of another vaccine, an anti-viral agent, a vaccine adjuvant, an anti-viral adjuvant, nitric oxide, and a nitric oxide releasing compound.

49. A method of treating a patient comprising: providing a vaccine to a patient; and
administering nitric oxide gas through inhalation to the patient.

50. The method of claim 49, wherein the patient has a viral infection.

51. The method of claim 49, further comprising administering to the patient one or more of another vaccine, an anti-viral agent, a vaccine adjuvant, an anti-viral adjuvant, nitric oxide, and a nitric oxide releasing compound.

52. A method of treating a patient comprising:
providing an anti-viral agent to a patient; and
administering nitric oxide gas through inhalation to the patient.

53. The method of claim 52, wherein the patient has a viral infection.

54. The method of claim 52, further comprising administering to the patient one or more of a vaccine, another anti-viral agent, a vaccine adjuvant, an anti-viral adjuvant, nitric oxide, and a nitric oxide releasing compound.

55. A method of treating a patient with a viral infection comprising:
administering nitric oxide gas at a concentration of at least about 100 ppm to the patient.

56. The method of claim 55, wherein the administering step is accomplished through inhalation of the nitric oxide gas into the respiratory tract of a patient.

57. The method of claim 55, wherein the administering is for at least about 100-400 breaths.

58. The method of claim 57, wherein the administering is from about 5-10 minutes to about 30 minutes every 4 hours.

59. The method of claim 55, wherein the nitric oxide gas has a concentration of about 160 ppm.

60. The method of claim 55, wherein the virus is selected from Influenza A, Influenza B, Avian Flu viruses, SARS (Corona) viruses, respiratory syncytial viruses, para-influenza viruses, Bovine Virus Diarrhea, HIV, and Rhinoviruses.

61. The method of claim 60, wherein the virus is an Avian Flu virus.

62. The method of claim 55, further comprising administering to the patient one or more of a vaccine, an anti-viral agent, a vaccine adjuvant, an anti-viral adjuvant, nitric oxide, and a nitric oxide releasing compound.

63. A method of preventing viral infection in a patient comprising:
administering nitric oxide gas at a concentration of at least about 100 ppm to the patient.

64. The method of claim 63, wherein the administering step is accomplished through inhalation of the nitric oxide gas into the respiratory tract of a patient.

65. The method of claim 63, wherein the administering is for at least about 100-400 breaths.

66. The method of claim 65, wherein the administering is from about 5-10 minutes to about 30 minutes every 4 hours.

67. The method of claim 63, wherein the nitric oxide gas has a concentration of about 160 ppm.

68. The method of claim 63, wherein the virus is selected from Influenza A, Influenza B, Avian Flu viruses, SARS (Corona) viruses, respiratory syncytial viruses, para-influenza viruses, Bovine Virus Diarrhea, HIV, and Rhinoviruses.

69. The method of claim 68, wherein the virus is an Avian Flu virus.

70. The method of claim 63, further comprising administering to the patient one or more of a vaccine, an anti-viral agent, a vaccine adjuvant, an anti-viral adjuvant, nitric oxide, and a nitric oxide releasing compound.
